(19) United States
(12) Patent Application Publication
(10) Pub. No.: US 2010/0179321 A1
(43) Pub. Date: Jul. 15, 2010

(54) SALT OF 5-[2-AMINO-4-(2-FURYL)PYRIMIDIN-5-YL]-1-METHYLPYRIDIN-2(1H)-ONE AND CRYSTAL THEREOF

(76) Inventors: Hiroshi Ishihara, Ibaraki (JP); Yoshiaki Sato, Ibaraki (JP)

Correspondence Address: BIRCH STEWART KOLASCH & BIRCH, PO BOX 747, FALLS CHURCH, VA, 22040-0747, US

(21) Appl. No.: 12/440,407

(22) PCT Filed: Sep. 6, 2007
(86) PCT No.: PCT/JP2007/067401

§ 371 Date: Mar. 6, 2009

(30) Foreign Application Priority Data
Sep. 7, 2006 (JP) 2006 243199

(51) Int. Cl. C07D 405/14

(52) U.S. Cl. 544/331

(57) ABSTRACT

The present invention provides a salt of 5-[2-amino-4-(2-furyl)pyrimidin-5-yl]-1-methylpyridin-2(1H)-one and a crystal thereof.

### TECHNICAL FIELD

The present invention relates to a salt of 5-[2-amino-4-(2-furyl)pyrimidin-5-yl]-1-methylpyridin-2(1H)-one, a compound effective for prevention and treatment for various diseases such as constipation, and a crystal thereof.

### BACKGROUND ART

5-[2-Amino-4-(2-furyl)pyrimidin-5-yl]-1-methylpyridin-2(1H)-one is a compound having adenosine receptor antagonism and being effective for prevention and treatment for various diseases such as constipation (see Patent Document 1). However, Patent document 1 does not specifically disclose a salt of 5-[2-amino-4-(2-furyl)pyrimidin-5-yl]-1-methylpyridin-2(1H)-one and a crystal thereof.

Patent Document 1: WO 03/035639

### DISCLOSURE OF THE INVENTION

### Problem to be Solved by the Invention

In general, the active ingredient of a medicament preferably has suitable physical properties such as being excellent in solubility. Thus, the object of the present invention is to provide a salt of 5-[2-amino-4-(2-furyl)pyrimidin-5-yl]-1-methylpyridin-2(1H)-one and a crystal thereof excellent in solubility.

### Means for Solving the Problems

The present inventors have energetically conducted studies and have consequently completed the present invention by finding that a salt of 5-[2-amino-4-(2-furyl)pyrimidin-5-yl]-1-methylpyridin-2(1H)-one disclosed in Patent Document 1 and a crystal thereof have suitable solubility.

Specifically, the present invention provides the following [1] to [14].

[1] a salt of 5-[2-amino-4-(2-furyl)pyrimidin-5-yl]-1-methylpyridin-2(1H)-one.
[2] a crystal of a salt according to [1].
[3] the crystal according to [2], wherein the salt is an inorganic acid salt.
[4] the crystal according to [2], wherein the salt is an organic acid salt.
[5] the crystal according to [2] or [3], wherein the salt is hydrochloride, and the crystal has a diffraction peak at a diffraction angle (2θ±0.2°) of 8.5° and/or 16.7° in a powder X-ray diffraction.
[6] the crystal according to [2] or [3], wherein the salt is hydrobromate, and the crystal has a diffraction peak at a diffraction angle (2θ±0.2°) of 8.4° and/or 11.3° in a powder X-ray diffraction.
[7] the crystal according to [2] or [3], wherein the salt is sulfate, and the crystal has a diffraction peak at a diffraction angle (2θ±0.2°) of 10.0° and/or 11.5° in a powder X-ray diffraction.
[8] the crystal according to [2] or [3], wherein the salt is sulfate, and the crystal has a diffraction peak at a diffraction angle (2θ±0.2°) of 6.4° and/or 10.2° in a powder X-ray diffraction.
[9] the crystal according to [2] or [3], wherein the salt is phosphate, and the crystal has a diffraction peak at a diffraction angle (2θ±0.2°) of 11.2° and/or 17.4° in a powder X-ray diffraction.
[10] the crystal according to [2] or [3], wherein the salt is phosphate, and the crystal has a diffraction peak at a diffraction angle (2θ±0.2°) of 5.0° and/or 6.6° in a powder X-ray diffraction.
[11] the crystal according to [2] or [4], wherein the salt is methanesulfonate, and the crystal has a diffraction peak at a diffraction angle (2θ±0.2°) of 9.6°, 15.3° and/or 19.2° in a powder X-ray diffraction.
[12] the crystal according to [2] or [4], wherein the salt is ethanesulfonate, and the crystal has a diffraction peak at a diffraction angle (2θ±0.2°) of 7.6° and/or 15.4° in a powder X-ray diffraction.
[13] the crystal according to [2] or [4], wherein the salt is benzenesulfonate, and the crystal has a diffraction peak at a diffraction angle (2θ±0.2°) of 9.2° and/or 18.5° in a powder X-ray diffraction.
[14] the crystal according to [2] or [4], wherein the salt is p-toluenesulfonate, and the crystal has a diffraction peak at a diffraction angle (2θ±0.2°) of 6.3° and/or 8.0° in a powder X-ray diffraction.

### EFFECTS OF THE INVENTION

The salt of the present invention and the crystal thereof have excellent solubility and are suitable for using as an active ingredient of a preventing and therapeutic agent for various diseases such as constipation.

### BRIEF DESCRIPTION OF THE DRAWINGS

FIG. 1 is a drawing representing a powder X-ray diffraction pattern of the crystals obtained in Example 1.

FIG. 2 is a drawing representing a powder X-ray diffraction pattern of the crystals obtained in Example 2.

FIG. 3 is a drawing representing a powder X-ray diffraction pattern of the crystals obtained in Example 3.

FIG. 4 is a drawing representing a powder X-ray diffraction pattern of the crystals obtained in Example 4.

FIG. 5 is a drawing representing a powder X-ray diffraction pattern of the crystals obtained in Example 5.

FIG. 6 is a drawing representing a powder X-ray diffraction pattern of the crystals obtained in Example 6.

FIG. 7 is a drawing representing a powder X-ray diffraction pattern of the crystals obtained in Example 7.

FIG. 8 is a drawing representing a powder X-ray diffraction pattern of the crystals obtained in Example 8.

FIG. 9 is a drawing representing a powder X-ray diffraction pattern of the crystals obtained in Example 9.

FIG. 10 is a drawing representing a powder X-ray diffraction pattern of the crystals obtained in Example 10.

FIG. 11 is a drawing representing a powder X-ray diffraction pattern of the crystal form A obtained in Preparation Example 7.

FIG. 12 is a drawing representing a powder X-ray diffraction pattern of the crystal form B obtained in Preparation Example 8.

FIG. 13 is a drawing showing results of solubility test.

### BEST MODES FOR CARRYING OUT THE INVENTION

Salt of the Present Invention

The salt of the present invention is a salt of 5-[2-amino-4-(2-furyl)pyrimidin-5-yl]-1-methylpyridin-2 (1H)-one.

The “salt” according to the present invention is not particularly limited as long as it forms a salt with 5-[2-amino-4-(2-furyl)pyrimidin-5-yl]-1-methylpyridin-2(1H)-one and is pharmaceutically acceptable one. Examples thereof include inorganic acid salts, organic acid salts, inorganic base salts, organic base salts and acidic or basic amino acid salts.

Preferable examples of the inorganic acid salts include hydrochloride, hydrobromate, sulfate, nitrate and phosphate. Preferable examples of the organic acid salts include acetate, succinate, fumarate, maleate, tartrate, citrate, lactate, stearate, benzoate, methanesulfonate, ethanesulfonate, benzenesulfonate and p-toluenesulfonate.

Preferable examples of the inorganic base salts include: alkali metal salts such as sodium salt and potassium salt; alkaline-earth metal salts such as calcium salt and magnesium salt; and aluminum salt and ammonium salt. Preferable examples of the organic base salts include diethylamine salt, diethanolamine salt, meglumine salt and N,N′-dibenzylethylenediamine salt.

Preferable examples of the acidic amino acid salts include aspartate and glutamate. Preferable examples of the basic amino acid salts include arginine salt, lysine salt and ornithine salt.

Crystal of the Present Invention

The crystal of the present invention is a crystal of the salt of the present invention. The crystal of the present invention encompasses, but not particularly limited to, the followings.

(1) hydrobromate characterized by having a diffraction peak at a diffraction angle (2θ±0.2°) of 8.4° and/or 11.3° in a powder X-ray diffraction.
(2) hydrochloride characterized by having a diffraction peak at a diffraction angle (2θ±0.2°) of 8.5° and/or 16.7° in a powder X-ray diffraction.
(3) benzenesulfonate characterized by having a diffraction peak at a diffraction angle (2θ±0.2°) of 9.2° and/or 18.5° in a powder X-ray diffraction.
(4) methanesulfonate characterized by having a diffraction peak at a diffraction angle (2θ±0.2°) of 9.6°, 15.3° and/or 19.2° in a powder X-ray diffraction.
(5) sulfate characterized by having a diffraction peak at a diffraction angle (2θ±0.2°) of 10.0° and/or 11.5° in a powder X-ray diffraction.
(6) sulfate characterized by having a diffraction peak at a diffraction angle (2θ±0.2°) of 6.4° and/or 10.2° in a powder X-ray diffraction.
(7) phosphate characterized by having a diffraction peak at a diffraction angle (2θ±0.2°) of 11.2° and/or 17.4° in a powder X-ray diffraction.
(8) phosphate characterized by having a diffraction peak at a diffraction angle (2θ±0.2°) of 5.0° and/or 6.6° in a powder X-ray diffraction.
(9) ethanesulfonate characterized by having a diffraction peak at a diffraction angle (2θ±0.2°) of 7.6° and/or 15.4° in a powder X-ray diffraction.
(10) p-toluenesulfonate characterized by having a diffraction peak at a diffraction angle (2θ±0.2°) of 6.3° and/or 8.0° in a powder X-ray diffraction.

Since the diffraction angle (2θ) in a powder X-ray diffraction may have a diffraction angle error in the range of ±0.2°, the values for the diffraction angle must be interpreted as including values within a range of ±0.2°. Thus, the present invention encompasses not only crystals whose peak diffraction angle in a powder X-ray diffraction matches exactly, but also crystals whose peak diffraction angle matches with an error of ±0.2°.

Specifically, taking the crystal of hydrobromate as an example, throughout the present specification, “having a diffraction peak at a diffraction angle (2θ±0.2°) of 8.4°” means “having a diffraction peak at a diffraction angle (2θ) in the range of 8.2°-8.6°”, and “having a diffraction peak at a diffraction angle (2θ±0.2°) of 11.3°” means “having a diffraction peak at a diffraction angle (2θ) in the range of 11.1°-11.5°”. Moreover, “having a diffraction peak at a diffraction angle (2θ±0.2°) of 8.4° and/or 11.3°” means having at least one of the above diffraction peaks.

Process for Preparing the Crystal of the Present Invention

The crystal of the present invention can be prepared as follows: 5-[2-amino-4-(2-furyl)pyrimidin-5-yl]-1-methylpyridin-2(1H)-one and a reagent (inorganic acid, organic acid, inorganic base, organic base, acidic or basic amino acid, etc.) corresponding to the salt are heated and dissolved in a solvent and then crystallized by gradual cooling. 5-[2-Amino-4-(2-furyl)pyrimidin-5-yl]-1-methylpyridin-2(1H)-one used for crystallization may be in any form. Specifically, it may be a hydrate or anhydrate, an amorphous or crystalline (including combinations of multiple crystal forms) compound, or a mixture thereof. 5-[2-Amino-4-(2-furyl)pyrimidin-5-yl]-1-methylpyridin-2(1H)-one can be prepared by the method disclosed in Patent Document 1, and also by the method described in Preparation Examples 1 to 8 below.

The solvent used for crystallization is not particularly limited as long as it dissolves starting materials to some extent. Examples thereof can include a single solvent or a mixed solvent of two or more selected from the group consisting of an alcoholic solvent such as methanol, ethanol, n-propanol and i-propanol (hereinafter referred to as “IPA”), an ether-based solvent such as tetrahydrofuran (hereinafter referred to as “THF”), dimethylsulfoxide and water. Preferred solvents are a single solvent of an alcoholic solvent, a mixed solvent of water and an alcoholic solvent or a mixed solvent of water and an ether-based solvent.

The amount of solvent used may be appropriately selected between the minimum amount in which the starting materials will dissolve by heating and the maximum amount at which the yield of the crystals is not significantly reduced, and it is preferably a 10-30 fold amount, in terms of the volume ratio with respect to the weight of the starting materials.

The temperature for dissolution of the starting materials may be appropriately selected depending on the solvent, but it is preferably from 70° C. to heating reflux temperature. The gradual cooling during crystallization is preferably carried out while suitably adjusting the cooling rate in consideration of the effect on quality and particle size of the crystals, preferably to a rate of 10° C. to 30° C./hr or slower, more preferably to a rate of 5° C. to 10° C./hr or slower, most preferably to a rate of 5° C./hr or slower.

The precipitated crystals may be separated by an ordinary filtration procedure, washed with an appropriate solvent if necessary, and then dried to give the desired crystals. The solvent used for washing of the crystals is generally the same as the crystallization solvent. The crystals can be dried by merely standing in air or by heating. The drying may be carried out under airflow or under reduced pressure.

Pharmaceutical Composition Comprising the Salt of the Present Invention

The salt of the present invention can be used as an active ingredient of a therapeutic agent for constipation in a similar fashion as the method disclosed in Patent Document 1.

The salt of the present invention may be formulated by an ordinary method into tablets, powders, fine powders, granules, coated tablets, capsules, syrups, lozenges, inhalants, suppositories, injections, ointments, eye ointments, eye drops, nose drops, ear drops, cataplasms, lotions or the like. For formulation there may be employed commonly used excipients, binders, lubricants, coloring agents, taste correctives and, if necessary, stabilizers, emulsifiers, absorption accelerators, surfactants, pH adjustors, antiseptics, antioxidants and the like, while other components ordinarily used as raw materials for medicament formulation may also be added according to common procedures.

Examples of such components include: animal or vegetable oils such as soybean oil, beef tallow and synthetic glycerides; hydrocarbons such as liquid paraffin, squalane and solid paraffin; ester oils such as octyldodecyl myristate and isopropyl myristate; higher alcohols such as cetostearyl alcohol and behenyl alcohol; silicone resins; silicone oils; surfactants such as polyoxyethylene fatty acid ester, sorbitan fatty acid ester, glycerin fatty acid ester, polyoxyethylenesorbitan fatty acid ester, polyoxyethylene hydrogenated castor oil and polyoxyethylene-polyoxypropylene block copolymer; water-soluble polymers such as hydroxyethylcellulose, polyacrylic acid, carboxyvinyl polymer, polyethylene glycol, polyvinylpyrrolidone and methylcellulose; lower alcohols such as ethanol and isopropyl alcohol; polyhydric alcohols such as glycerin, propylene glycol, dipropylene glycol and sorbitol; sugars such as glucose and sucrose; inorganic powders such as silicic anhydride, magnesium aluminum silicate and aluminum silicate; and purified water.

As the excipients there may be used lactose, corn starch, white soft sugar, glucose, mannitol, sorbit, crystalline cellulose, silicon dioxide and the like. As the binders there may be used polyvinyl alcohol, polyvinyl ether, methylcellulose, ethylcellulose, gum Arabic, tragacanth gum, gelatin, shellac, hydroxypropylmethylcellulose, hydroxypropylcellulose, polyvinylpyrrolidone, polypropylene glycol-polyoxyethylene block polymer, meglumine and the like. As the disintegrators there may be used starch, agar, gelatin powder, crystalline cellulose, calcium carbonate, sodium hydrogencarbonate, calcium citrate, dextrin, pectin, carboxymethylcellulose calcium and the like. As the lubricants there may be used magnesium stearate, talc, polyethylene glycol, silica, hydrogenated vegetable oils and the like. As the coloring agents there may be used those approved for addition to pharmaceuticals. As the taste correctives there may be used cocoa powder, menthol, aromatic powder, peppermint oil, camphor, cinnamon powder and the like.

For production of an oral preparation, the salt of the present invention may be combined with an excipient and, if necessary, a binder, disintegrator, lubricant, coloring agent, taste corrective or the like and then made into powders, fine powders, granules, tablets, coated tablets or capsules by an ordinary method.

These tablets or granules may be appropriately coated by sugar-coating or, if necessary, other appropriate coatings.

For production of a liquid preparation such as a syrup or pharmaceutical preparation for injection, the salt of the present invention may be combined with a pH adjustor, solubilizer, isotonizing agent or the like, and if necessary, a dissolving aid, stabilizer or the like, and formulated by an ordinary method.

The method of producing an external preparation is not particularly limited, and may be according to an ordinary method. Specifically, as base materials for such a pharmaceutical preparation there may be used various materials ordinarily employed for pharmaceuticals, quasi drugs, cosmetics and the like. Specific examples of the base materials that may be used include materials such as animal and vegetable oils, mineral oils, ester oils, waxes, higher alcohols, fatty acids, silicone oils, surfactants, phospholipids, alcohols, polyhydric alcohols, water-soluble polymers, clay minerals and purified water. If necessary, there may also be added pH adjustors, antioxidants, chelating agents, antiseptic/mildew resistant agents, coloring agents, aromatics and the like, although the base materials for an external preparation according to the present invention are not limited to these. If necessary, there may also be included components such as circulation promoters, bactericidal agents, antiflash agents, cell activators, vitamins, amino acids, humectants and keratolytic agents. The amounts of such base materials added are the amounts which give concentrations indicated for production of ordinary external preparations.

The form of administration of the salt of the present invention is not particularly limited, and may be oral administration or parenteral administration by an ordinarily employed method. For example, the salt may be administered after formulation into tablets, powder, granules, capsules, syrups, lozenges, inhalants, suppositories, injections, ointments, eye ointments, eye drops, nose drops, ear drops, cataplasms, lotions or the like. The dosage of a pharmaceutical according to the present invention may be appropriately selected depending on patient age, gender, body weight, severity of symptoms, particular type of condition, and on the type of dosage form or salt. For example, it will generally be administered once or divided over several times at about 30 μg to 10 g, preferably 100 μg to 5 g, more preferably 100 μg to 100 mg per day for adults in the case of oral administration or at about 30 μg to 1 g, preferably 100 μg to 500 mg, more preferably 100 μg to 30 mg per day in the case of injection.

### EXAMPLES

### Preparation Example 1

### Synthesis of (6-methoxypyridin-3-yl)methanol (2)

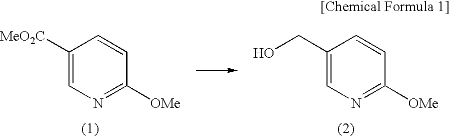

To a solution of methyl-6-methoxynicotinate (1) (650 g, 3.89 mol) in t-butyl methyl ether (hereinafter abbreviated as “MTBE”) (6.5 L) cooled in an ice bath, sodium bis(2-methoxyethoxy) aluminum hydride (65% solution in toluene, 1.45 kg, 4.67 mol) was added under a nitrogen atmosphere over a period of 1.3 hours. After stirring for 20 minutes, a 3.5 N aqueous solution of sodium hydroxide (2.6 L) was added to the reaction mixture while keeping the temperature 15° C. or below. The reaction mixture was stirred at 32° C. for 45 minutes. Then, the organic layer was separated, and the aqueous layer was re-extracted with MTBE (2.3 L). The organic layers were combined and concentrated under reduced pressure to dryness. Toluene (1.3 L) was added to the residue, followed by azeotropic distillation. Azeotropic distillation with toluene (1.3 L) was repeated three times to give 597 g of the title compound as a pale yellow oil (yield 100%).

¹H-NMR (CDCl₃) δ (ppm): 8.11 (1H, d, J=2.4 Hz), 7.62 (1H, dd, J=2.4 Hz, 8.8 Hz), 6.75 (1H, d, J=8.8 Hz), 4.62 (2H, s), 3.93 (3H, s)

### Preparation Example 2

### Synthesis of 5-chloromethyl-2-methoxypyridine (3)

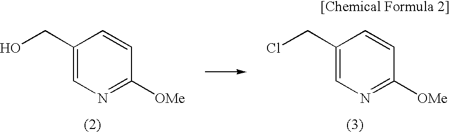

To a solution of (6-methoxypyridin-3-yl)methanol (2) (537.8 g, 3.86 mol) obtained in Preparation Example 1 in dimethylformamide (1.6 L), thionyl chloride (310 mL, 4.25 mol) was added dropwise under a nitrogen atmosphere over a period of 1.3 hours while cooling in an ice bath. After stirring for 1 hour while cooling in an ice bath, toluene (5.4 L) and a 2 N aqueous solution of sodium hydroxide (5.4 L) were added successively to the reaction mixture at 23° C. or below. The reaction mixture was stirred for about 10 minutes. Then, the aqueous layer was separated, and the organic layer was washed with water (2.7 L). The organic layer was concentrated under reduced pressure to dryness. Toluene (1.0 L) was added to the residue, followed by azeotropic distillation to give 618.8 g of the title compound as a pale yellow oil (content 556.3 g, yield 91.4%).

¹H-NMR (CDCl₃) δ (ppm): 8.15 (1H, d, J=2.4 Hz), 7.63 (1H, dd, J=2.4 Hz, 8.4 Hz), 6.75 (1H, d, J=8.4 Hz), 4.55 (2H, s), 3.94 (3H, s)

### Preparation Example 3

### Synthesis of furan-2-yl-morpholin-4-yl-acetonitrile (5)

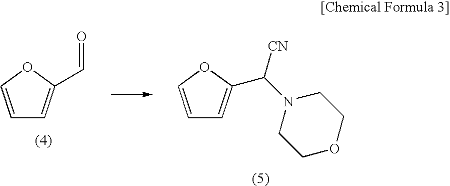

A solution of furfural (4) (550 g, 5.72 mol) in toluene (5.5 L) was cooled to 8° C., and an aqueous solution (1.1 L water) of potassium cyanide (384.6 g, 5.72 mol) was added thereto over a period of 7 minutes.

Next, to the reaction mixture, an aqueous solution (1.65 L water) of p-toluenesulfonic acid monohydrate (1143.0 g, 6.01 mol) was added over a period of 20 minutes, and the reaction mixture was further stirred for 1 hour. To the reaction mixture, a solution of morpholine (997 g, 11.45 mol) in toluene (100 mL) was added over a period of 8 minutes, followed by stirring for 2.5 hours in a water bath at about 20° C. The aqueous layer was separated, and the organic layer was washed with water (2.75 L) and then concentrated under reduced pressure to dryness to give 1028.7 g of the title compound as a reddish-brown oil (content 90.2%, yield 84.3%).

¹H-NMR (CDCl₃) δ (ppm): 7.47 (1H, brs), 6.57 (1H, d, J=3.2 Hz), 6.41 (1H, dd, J=3.2 Hz, 1.6 Hz), 4.85 (1H, s), 4.43 (4H, m), 4.31 (4H, m)

### Preparation Example 4

### Synthesis of 1-furan-2-yl-2-(6-methoxy-pyridin-3-yl)-ethanone (7)

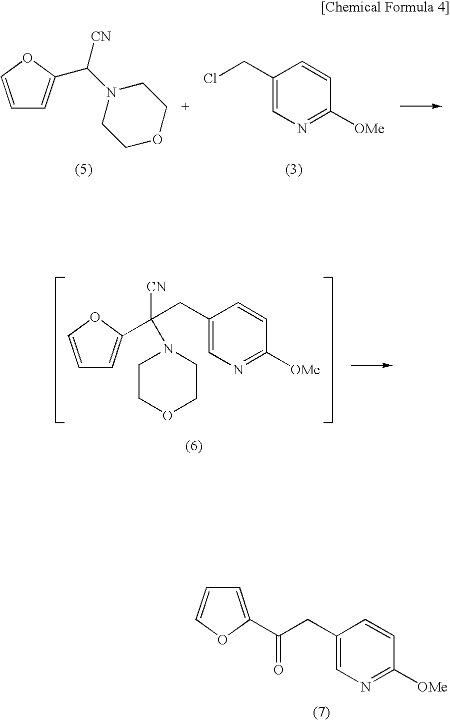

A solution of furan-2-yl-morpholin-4-yl-acetonitrile (5) (818.0 g, content 737.9 g, 3.84 mol) obtained in Preparation Example 3 and 5-chloromethyl-2-methoxypyridine (3) (611.8 g, content 550.0 g, 3.49 mol) obtained in Preparation Example 2 in toluene (4.4 L) was cooled to −15° C. A solution of potassium-t-butoxide (508.9 g, 4.54 mol) in tetrahydrofuran (4.4 L) was added thereto at −5° C. or below over a period of 72 minutes, and the reaction mixture was further stirred for 1.5 hours.

Next, to the reaction mixture, a 6 N aqueous solution of hydrochloric acid (4.4 L) was added, followed by heating to 70° C. and stirring for 2 hours. The reaction mixture was cooled to 5° C., and a 3 N aqueous solution of sodium hydroxide (3.0 L) was added at 20° C. or below. The organic layer was separated, and the aqueous layer was re-extracted with toluene (6.0 L). The organic layers were combined and concentrated under reduced pressure to dryness to give 828.5 g of the title compound as a brown oil (content 647.8 g, yield 85.5%).

### 2-Furan-2-yl-3-(6-methoxy-pyridin-3-yl)-2-morpholin-4-yl-propionate (6)

¹H-NMR (CDCl₃) δ (ppm): 7.71 (1H, d, J=2.4 Hz), 7.48 (1H, d, J=1.6 Hz), 7.11 (1H, dd, J=2.4 Hz, 8.4 Hz), 6.56 (1H, d, J=8.4 Hz), 6.27 (2H, m), 3.87 (3H, s), 3.80 (4H, m), 3.38 (1H, d, J=13.2), 3.26 (1H, d, J=13.2), 2.78-2.81 (2H, m), 2.45-2.78 (2H, m)

### 1-Furan-2-yl-2-(6-methoxy-pyridin-3-yl)-ethanone (7)

¹H-NMR (CDCl₃) δ (ppm): 8.08 (1H, d, J=2.4 Hz), 7.61 (1H, d, J=1.7 Hz), 7.53 (1H, dd, J=2.4 Hz, 8.2 Hz), 7.24 (1H, d, J=3.6 Hz), 6.71 (1H, d, J=8.2 Hz), 6.55 (1H, dd, 1=1.7 Hz, 3.6 Hz), 4.05 (2H, s), 3.91 (3H, s)

### Preparation Example 5

### Synthesis of 5-(2-furan-2-yl-2-oxo-ethyl)-1-methyl-1H-pyridin-2-one (8)

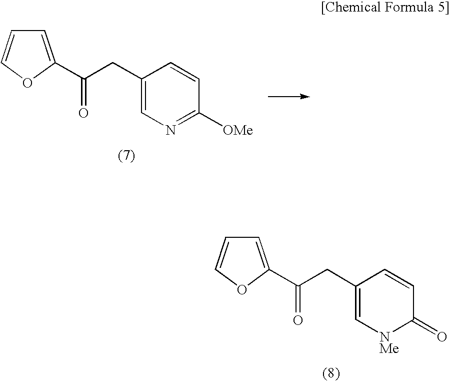

To a solution of 1-furan-2-yl-2-(6-methoxy-pyridin-3-yl)-ethanone (7) (800.0 g, content 625.6 g, 2.88 mol) obtained in Preparation Example 4 in N-methyl-2-pyrrolidine (NMP) (1.88 L), iodomethane (122.6 g, 0.86 mol) was added, and the reaction mixture was stirred at 100° C. for 3 hours and then at room temperature for 17.5 hours. To the reaction mixture, MTBE (6.6 L) was added dropwise over a period of 77 minutes, followed by stirring for 1 hour while cooling in an ice bath. The precipitated crystals were collected by filtration, washed with MTBE (2.0 L), and then dried under reduced pressure at 50° C. for 3 hours to give 692.0 g of a crude product of the title compound as a dark brown powder (content 536.4 g, yield 85.7%).

To the obtained crude product (682.0 g, content 528.7 g, 2.43 mol), 1,2-dimethoxyethane (hereinafter abbreviated as “DME”) (7.93 L) and water (0.68 L) were added, followed by heating and stirring at 80° C. for 75 minutes. After confirming dissolution, stirring was continued overnight at 8° C. The precipitated crystals were collected by filtration, washed with DME (2.0 L), and then air-dried at 60° C. for 2.3 hours to give 468.46 g of the title compound as pale yellow crystals (content 462.8 g, yield 87.5%).

¹H-NMR (DMSO-d₆) δ (ppm): 8.02 (1H, d, J=1.6 Hz), 7.57 (2H, m), 7.30 (1H, dd, J=3.4 Hz, 9.2 Hz), 6.74 (1H, dd, J=1.6 Hz, 3.6 Hz), 6.33 (1H, d, J=9.2 Hz), 3.98 (2H, s), 3.38 (3H, s)

### Preparation Example 6

### Synthesis of 5-[2-amino-4-(2-furyl)pyrimidin-5-yl]-1-methylpyridin-2(1H)-one (10)

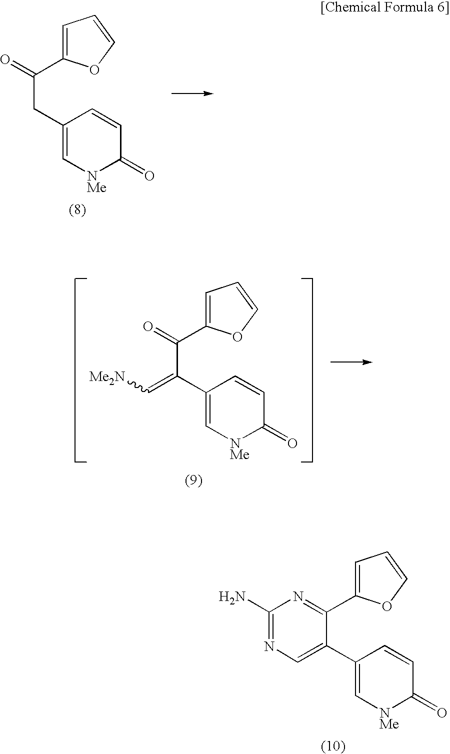

To 5-(2-furan-2-yl-2-oxo-ethyl)-1-methyl-1H-pyridin-2-one (8) (402.0 g, content 397.6 g, 1.83 mol) obtained in Preparation Example 5, dimethylformamide (0.4 L) and N,N-dimethylformamide dimethyl acetal (654.4 g, 5.49 mol) were added, and the reaction mixture was stirred at 60° C. for 10.5 hours and then at room temperature for 13.5 hours. To the reaction mixture, guanidine hydrochloride (524.56 g, 5.49 mol) and 1,8-diazabicyclo[5.4.0]undec-7-en (DBU) (821 mL, 5.49 mol) were added, and the reaction mixture was stirred at 70° C. for 7.8 hours. Next, to the reaction mixture, 2-propanol (12.0 L) was added, followed by stirring for 2 hours in an ice bath. The precipitated crystals were collected by filtration, washed with 2-propanol (1.0 L), and then air-dried at 60° C. for 13 hours to give 424.9 g of the title compound as pale yellow crystals (content 413.0 g, yield 84.1%).

### 5-[2-Dimethylamino-1-(furan-2-carbonyl)-vinyl]-1-methyl-1H-pyridin-2-on e (9)

¹H-NMR (CDCl₃) δ (ppm): 7.77 (1H, s), 7.45 (1H, d, J=2.0 Hz), 7.26 (1H, dd, J=2.4 Hz, 9.2 Hz), 7.14 (1H, dd, J=2.4 Hz), 6.60 (1H, d, J=9.2 Hz), 6.50 (1H, J=3.2 Hz), 6.37 (1H, J=3.2 Hz), 3.55 (3H, s), 2.93 (6H, brs)

### 5-[2-Amino-4-(2-furyl)pyrimidin-5-yl]-1-methylpyridin-2(1H)-one (10)

¹H-NMR (DMSO-d₆) δ (ppm): 8.13 (1H, s), 7.75 (1H, dd, J=0.7 Hz, 1.4 Hz), 7.72 (1H, d, J=2.4 Hz), 7.20 (1H, dd, J=2.4 Hz, 9.0 Hz), 6.78 (2H, brs), 6.72 (1H, d, J=3.5 Hz), 6.56 (1H, m), 6.36 (1H, d, J=9.0 Hz), 3.44 (3H, s)

### Preparation Example 7

### Preparation of crystal form A

To 30 g of 5-[2-amino-4-(2-furyl)pyrimidin-5-yl]-1-methylpyridin-2(1H)-one (10) obtained in Preparation Example 6, 30 mL of 2-propanol and 150 mL of water were added, followed by heating and stirring for 25 minutes in an oil bath at 90° C. After confirming the absence of solid, filtration with heating was performed, and the filtrate was heated and stirred at 70° C. for about 30 minutes. The reaction mixture was then heated and stirred for 1.3 hours at an external temperature of 55° C., and subsequently stirred for 2.3 hours at an external temperature of 45 to 40° C. Precipitation of crystals was confirmed at an internal temperature of about 47° C. The reaction mixture was further stirred at 30° C. for about 40 minutes, at room temperature for 1 hour and at 4° C. for 1.6 hours, and then the crystals were collected by filtration. The crystals were washed three times with 20 mL of 2-propanol and dried at 60° C. for 10.5 hours to give 19.9 g of crystals of 5-[2-amino-4-(2-furyl)pyrimidin-5-yl]-1-methylpyridin-2(1H)-one (hereinafter referred to as “crystal form A”).

### Preparation Example 8

### Preparation of crystal form B

To 10 g of 5-[2-amino-4-(2-furyl)pyrimidin-5-yl]-1-methylpyridin-2(1H)-one (10) obtained in Preparation Example 6, 10 mL of 2-propanol and 50 mL of water were added, followed by heating and stirring for about 30 minutes at an external temperature of 85° C. After confirming dissolution, the solution was cooled in an ice bath and stirred for 1.5 hours, and the crystals were collected by filtration. The crystals were washed twice with 10 mL of 2-propanol and dried at 60° C. for 10.5 hours to give 6.84 g of crystals of 5-[2-amino-4-(2-furyl)pyrimidin-5-yl]-1-methylpyridin-2(1H)-one (hereinafter referred to as “crystal form B”) whose crystal form is different from the crystal form A of Preparation Example 7.

### Example 1

### Preparation of 5-[2-amino-4-(2-furyl)pyrimidin-5-yl]-1-methylpyridin-2(1H)-one hydrobromate (11)

The crystal form A (about 300 mg) obtained in Preparation Example 7 was dissolved in THF (3 mL). Next, after adding hydrobromic acid (about 1 equivalent of the compound), 1 mL of water and 2 mL of IPA were added, and the reaction mixture was completely dissolved by heating to 90° C. The solution was gradually added to 10 mL of 2-propanol heated to 90° C., followed by standing at room temperature to give 5-[2-amino-4-(2-furyl)pyrimidin-5-yl]-1-methylpyridin-2(1H)-one hydrobromate (11).

### Example 2

### Preparation of 5-[2-amino-4-(2-furyl)pyrimidin-5-yl]-1-methylpyridin-2(1H)-one hydrochloride (12)

The crystal form A (about 300 mg) obtained in Preparation Example 7 was mixed with water (1.5 mL), IPA (1.5 mL) and hydrochloric acid (1.8 equivalent of the compound), and then the reaction mixture was completely dissolved by heating to 85° C. The solution was gradually added to 20 mL of IPA heated to 85° C., followed by standing at room temperature to give 5-[2-amino-4-(2-furyl)pyrimidin-5-yl]-1-methylpyridin-2(1H)-one hydrochloride (12).

### Example 3

### Preparation of 5-[2-amino-4-(2-furyl)pyrimidin-5-yl]-1-methylpyridin-2(1H)-one benzenesulfonate (13)

The crystal form A (about 300 mg) obtained in Preparation Example 7 was mixed with 500 μL of an aqueous solution of benzenesulfonic acid (about 1.8 equivalent of the compound), and then the reaction mixture was completely dissolved by heating to 80° C. The solution was gradually added to 5 mL of EtOH heated to 80° C., followed by standing at room temperature to give 5-[2-amino-4-(2-furyl)pyrimidin-5-yl]-1-methylpyridin-2(1H)-one benzenesulfonate (13).

### Example 4

### Preparation of 5-[2-amino-4-(2-furyl)pyrimidin-5-yl]-1-methylpyridin-2(1H)-one methanesulfonate (14)

The crystal form A (about 300 mg) obtained in Preparation Example 7 was mixed with methanesulfonic acid (about 1.1 equivalent of the compound) and 450 μL, of water, and then the reaction mixture was completely dissolved by heating to 80° C. The solution was gradually added to 7 mL of IPA heated to 80° C., followed by standing at room temperature and drying to give 5-[2-amino-4-(2-furyl)pyrimidin-5-yl]-1-methylpyridin-2(1H)-one methanesulfonate (14).

### Example 5

### Preparation of 5-[2-amino-4-(2-furyl)pyrimidin-5-yl]-1-methylpyridin-2(1H)-one sulfate (15)

The crystal form A (about 300 mg) obtained in Preparation Example 7 was mixed with sulfuric acid (about 1.1 equivalent of the compound) and 450 μL of water, and then the reaction mixture was completely dissolved by heating to 90° C. The solution was gradually added to 6 mL of THF heated to 90° C., followed by standing at room temperature to give 5-[2-amino-4-(2-furyl)pyrimidin-5-yl]-1-methylpyridin-2 (1H)-one sulfate 15).

### Example 6

### Preparation of 5-[2-amino-4-(2-furyl)pyrimidin-5-yl]-1-methylpyridin-2(1H)-one sulfate 16)

A compound (about 270 mg) having the same crystal form as the crystal form B obtained in Preparation Example 8 was mixed with 9 mL of n-propanol and 1 mL of water, and the reaction mixture was completely dissolved by heating to 90° C. using an oil bath. After adding sulfuric acid (about 0.5 equivalent of the compound) to this solution, heating was stopped, and the solution was left standing until the temperature reached room temperature. The obtained solid was filtered and dried at 60° C. to give 5-[2-amino-4-(2-furyl)pyrimidin-5-yl]-1-methylpyridin-2(1H)-one sulfate (16).

### Example 7

### Preparation of 5-[2-amino-4-(2-furyl)pyrimidin-5-yl]-1-methylpyridin-2(1H)-one phosphate (17)

The crystal form A (about 300 mg) obtained in Preparation Example 7 was mixed with phosphoric acid (about 2 equivalent of the compound), 300 μL of methanol and 2 mL of water, and then the reaction mixture was completely dissolved by heating to 90° C. The solution was gradually added to 12 mL of IPA heated to 90° C., followed by standing at room temperature to give 5-[2-amino-4-(2-furyl)pyrimidin-5-yl]-1-methylpyridin-2(1H)-one phosphate (17).

### Example 8

### Preparation of 5-[2-amino-4-(2-furyl)pyrimidin-5-yl]-1-methylpyridin-2(1H)-one phosphate (18)

The crystal form A (about 200 mg) obtained in Preparation Example 7 was mixed with phosphoric acid (about 0.67 equivalent of the compound), 500 μL of IPA, 200 μL of methanol and 700 μL of water, and then the reaction mixture was completely dissolved by heating to 85° C. The solution was gradually added to 8 mL of IPA heated to 90° C., followed by standing at room temperature to give 5-[2-amino-4-(2-furyl)pyrimidin-5-yl]-1-methylpyridin-2(1H)-one phosphate (18).

### Example 9

### Preparation of 5-[2-amino-4-(2-furyl)pyrimidin-5-yl]-1-methylpyridin-2(1H)-one ethanesulfonate (19)

The crystal form A (about 200 mg) obtained in Preparation Example 7 was mixed with ethanesulfonic acid (about 1.1 equivalent of the compound) and 300 μL of water, and then the reaction mixture was completely dissolved by heating to 85° C. The solution was gradually added to 5 mL of IPA heated to 85° C., followed by standing at room temperature to give 5-[2-amino-4-(2-furyl)pyrimidin-5-yl]-1-methylpyridin-2(1H)-one ethanesulfonate (19).

### Example 10

### Preparation of 5-[2-amino-4-(2-furyl)pyrimidin-5-yl]-1-methylpyridin-2(1H)-one p-toluenesulfonate (20)

The crystal form A (about 200 mg) obtained in Preparation Example 7 was mixed with (about 1.1 equivalent of the compound) and 500 μl, of water, and then the reaction mixture was completely dissolved by heating to 85° C. The solution was gradually added to 5 mL of IPA heated to 85° C., followed by standing at room temperature to give 5-[2-amino-4-(2-furyl)pyrimidin-5-yl]-1-methylpyridin-2(1H)-one p-toluenesulfonate (20).

Measurement of Powder X-ray Diffraction Pattern

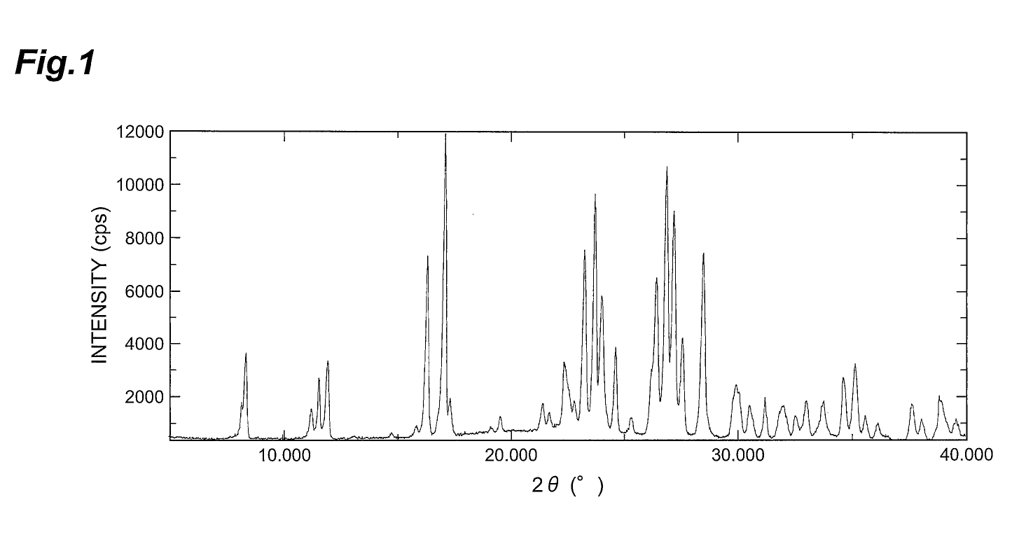

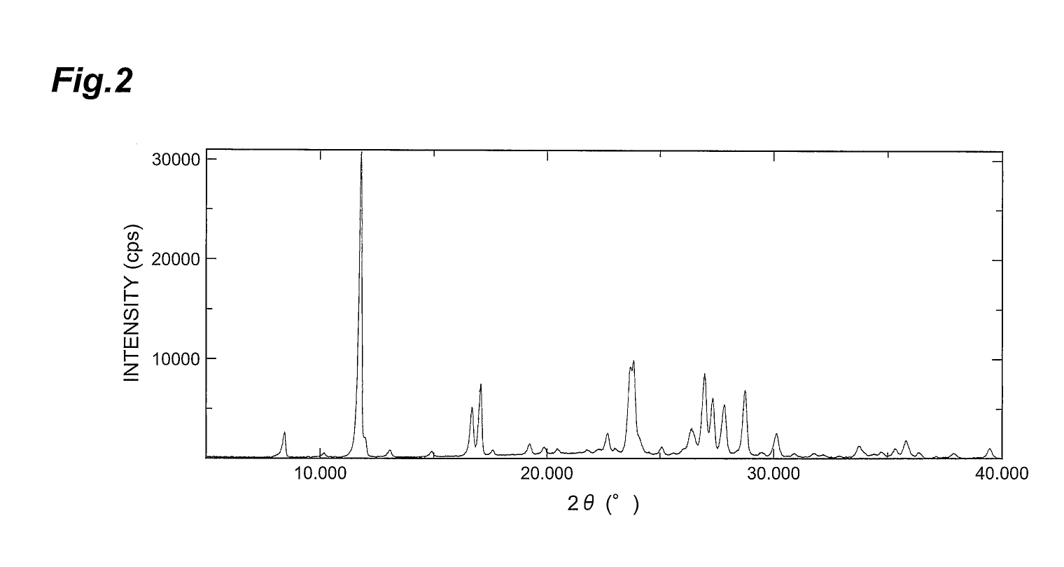

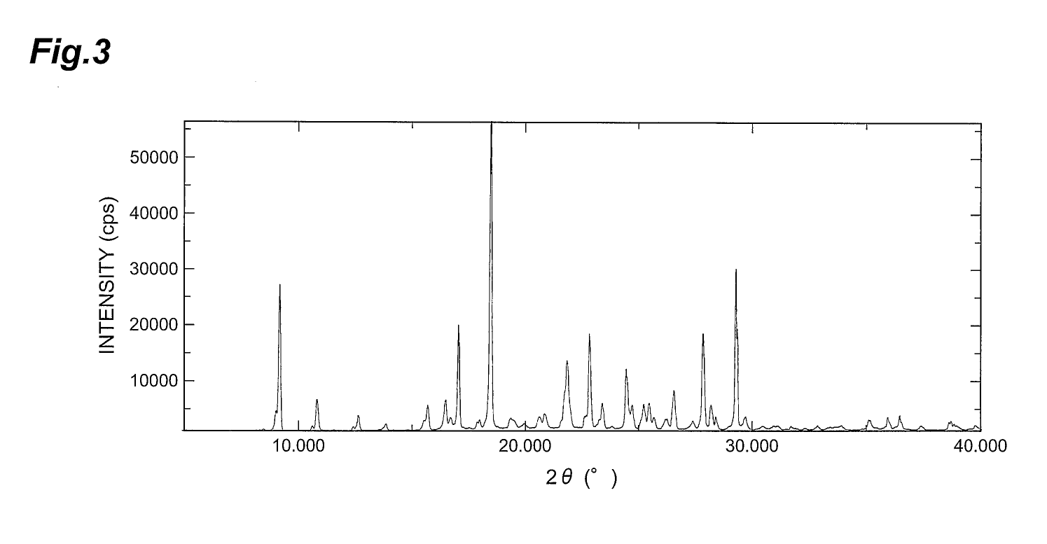

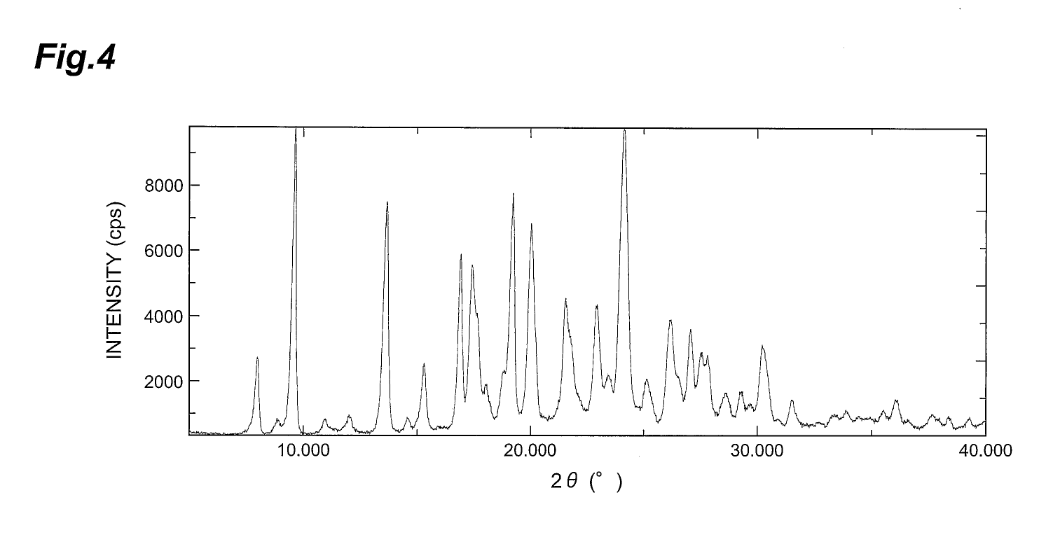

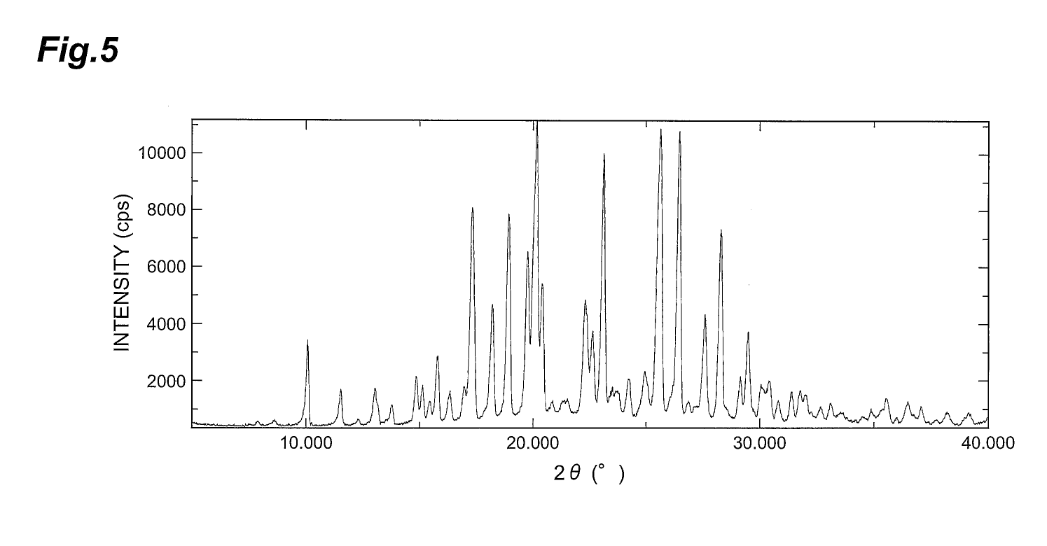

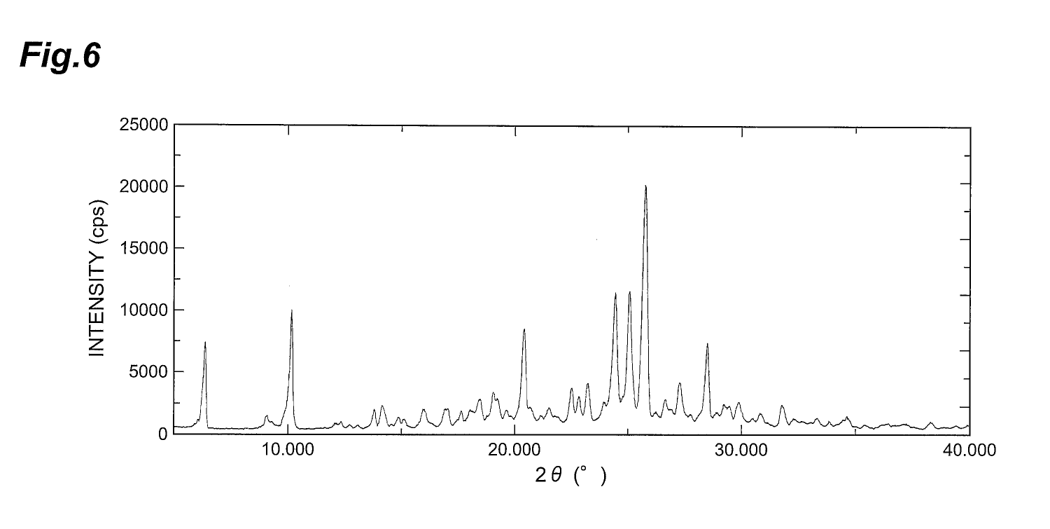

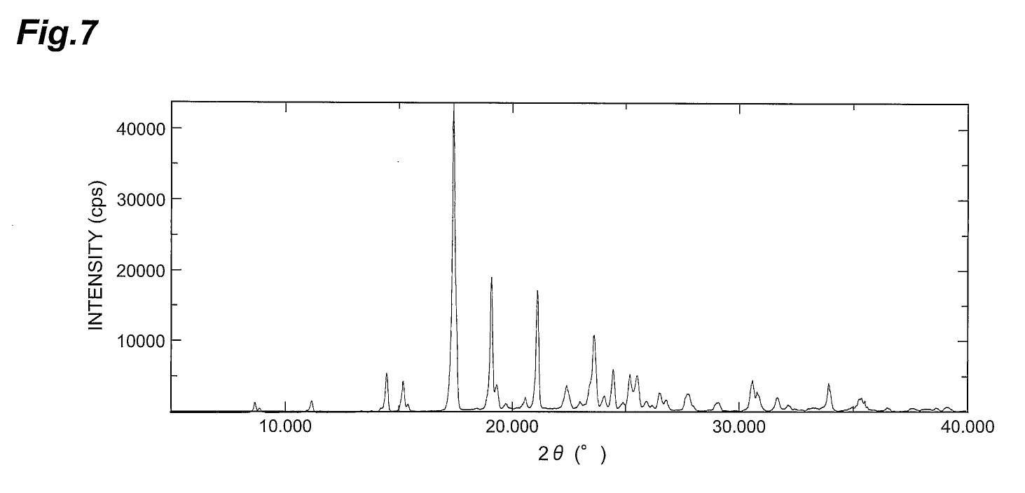

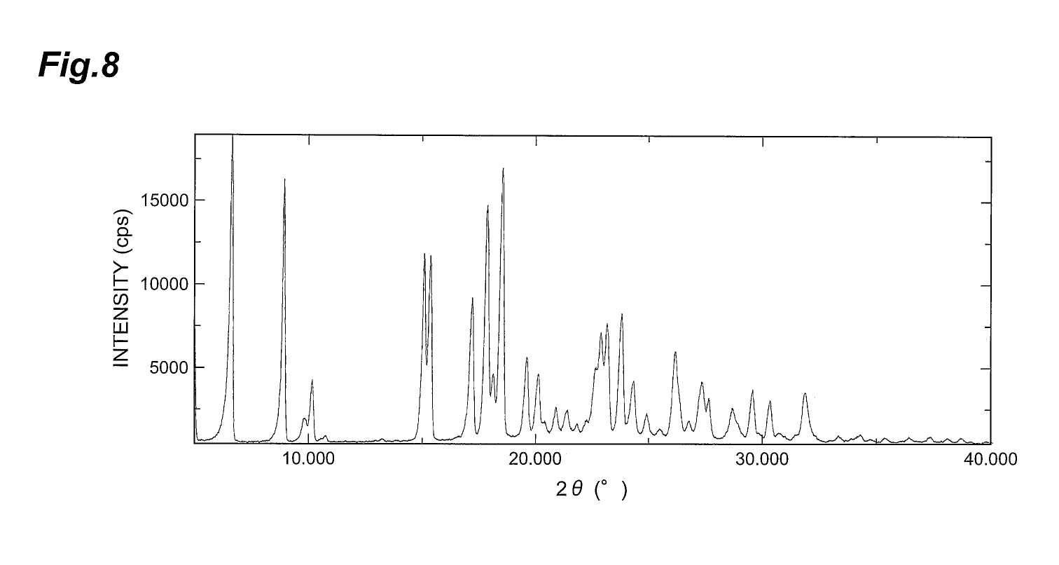

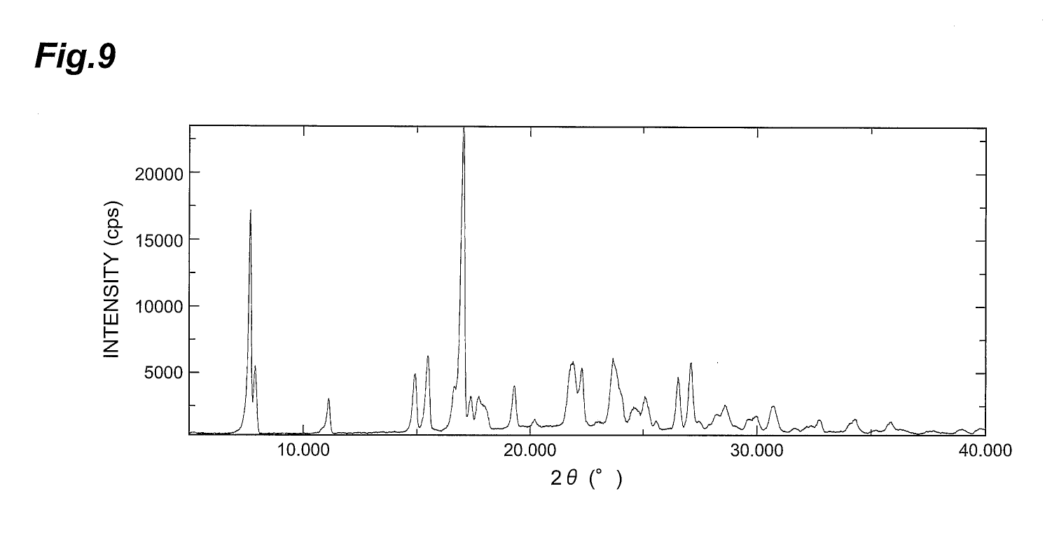

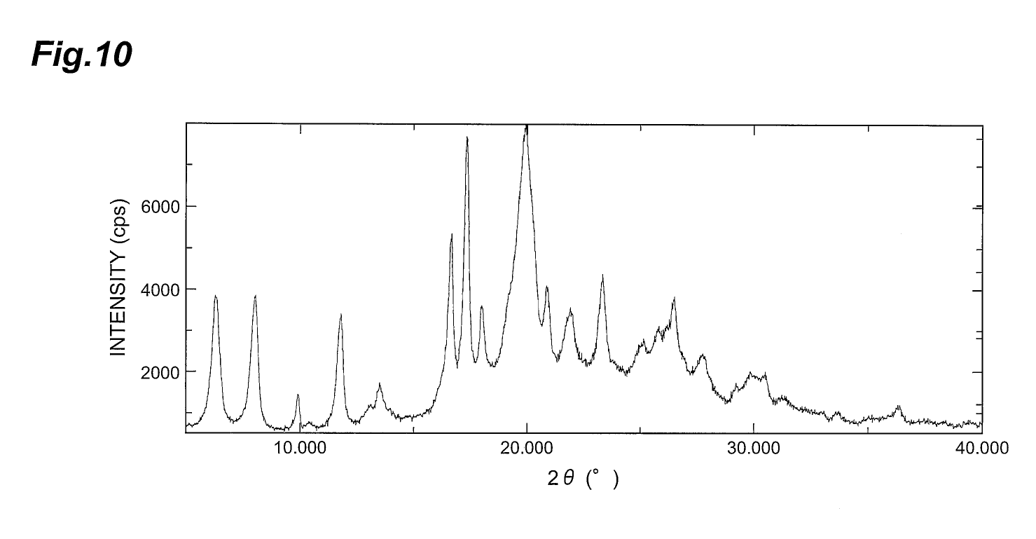

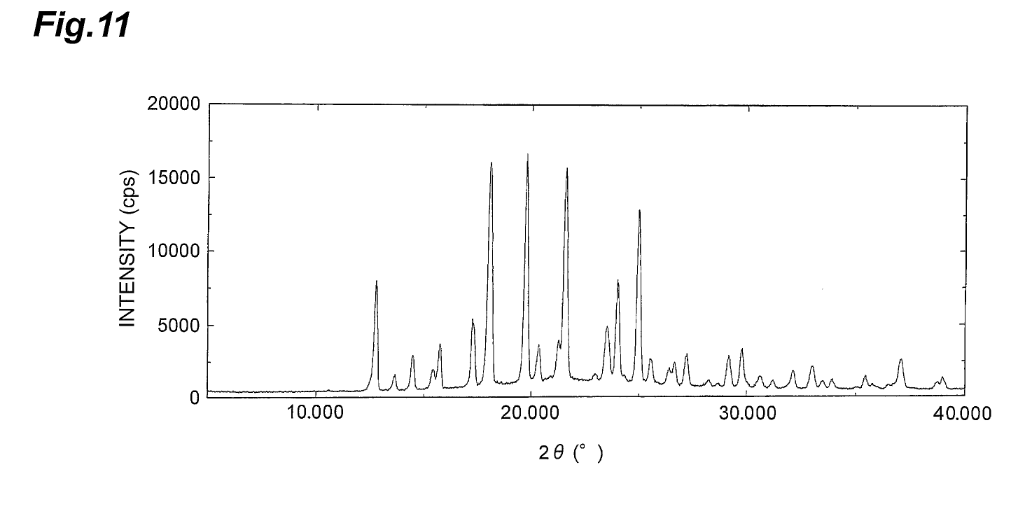

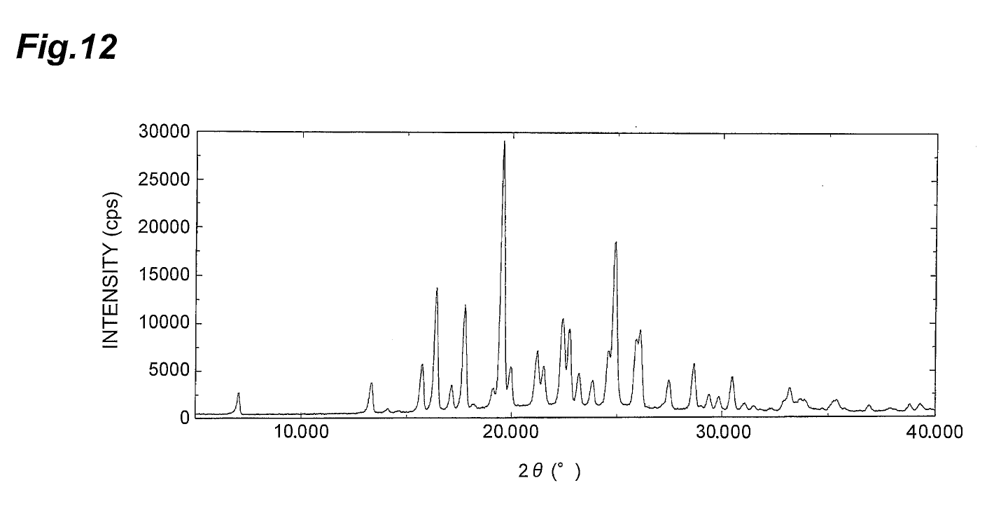

The powder X-ray diffraction patterns of the respective crystals obtained in Examples 1 to 10 and Preparation Examples 7 and 8 (hydrobromate (11), hydrochloride (12), benzenesulfonate (13), methanesulfonate (14), sulfate (15), sulfate (16), phosphate (17), phosphate (18), ethanesulfonate (19), p-toluenesulfonate (20), crystal form A and crystal form B) were measured under the following conditions. The powder X-ray diffraction patterns for the respective crystals of Examples 1 to 10 and Preparation Examples 7 and 8 are shown in FIGS. 1 to 12. The characteristic diffraction angle (2θ) peaks for the respective crystals are summarized in Table 1.

Target/tube current/tube voltage: Cu/40 kV/200 mA
Monochrometer: Full-automatic monochrometer
Counter: Scintillation counter
Scan speed: 2°/min
Scan step: 0.02°
Scanning axis: 2θ/θ
Scanning range: 5-40°
Divergence slit: 0.5°
Scattering slit: 0.5°
Receiving slit: 0.3 mm

TABLE 1

| Crystal | 2θ (°) |
|---|---|
| Hydrobromate (11) | 8.4, 11.3 |
| Hydrochloride (12) | 8.5, 16.7 |
| Benzenesulfonate (13) | 9.2, 18.5 |
| Methanesulfonate (14) | 9.6, 15.3, 19.2 |
| Sulfate (15) | 10.0, 11.5 |
| Sulfate (16) | 6.4, 10.2 |
| Phosphate (17) | 11.2, 17.4 |
| Phosphate (18) | 5.0, 6.6 |
| Ethanesulfonate (19) | 7.6, 15.4 |
| p-Toluenesulfonate (20) | 6.3, 8.0 |
| Crystal form A | 12.8, 18.1, 23.5 |
| Crystal form B | 7.0, 16.4 |

Solubility Test

About 70 mg of the respective crystals obtained in Examples 1 to 3, 5,7 to 10 and Preparation Example 8 (hydrobromate (11), hydrochloride (12), benzenesulfonate (13), sulfate (15), phosphate (17), phosphate (18), ethanesulfonate (19), p-toluenesulfonate (20) and crystal form B) was used to evaluate solubility in Solution 2 of the disintegration test in the Japanese Pharmacopoeia (JP) 14th Edition under the following conditions.

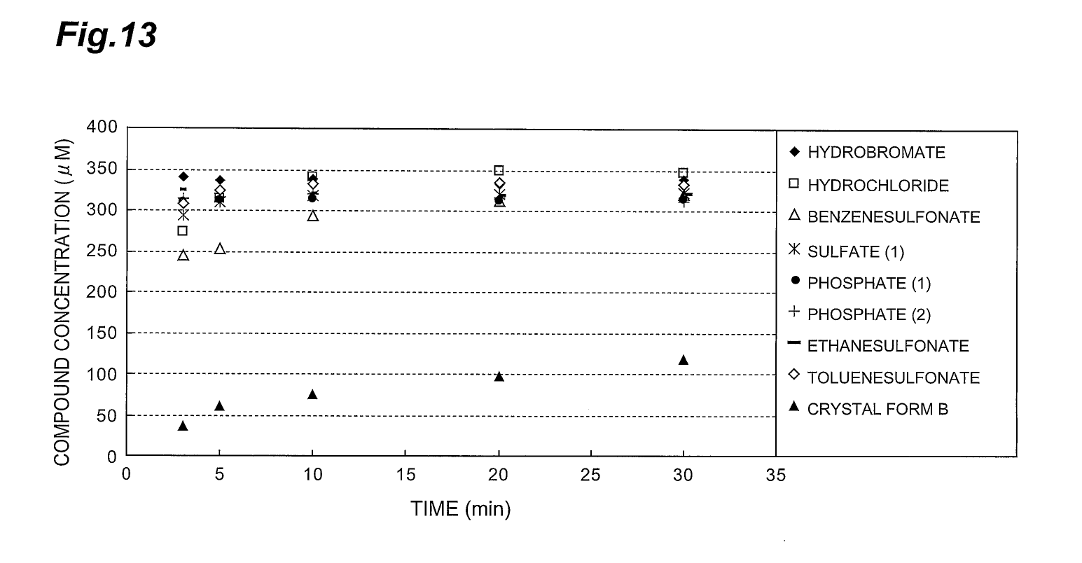

First, 500 mL of JP Solution 2 kept at 37° C. was stirred at 50 rpm with a paddle (DISSOLUTION TESTER by Toyama Sangyo Co., Ltd.). After adding the respective crystals (adding in the amount which gave the final concentration of about 350 μM when completely dissolved), sample was collected periodically. The solutions were separately filtered through filters (0.2 μm), and then the compound concentrations in the solutions were measured by the HPLC method. HPLC conditions were as follows. The obtained results are shown in FIG. 13.

(HPLC Conditions)

Column: CAPCELL PAK C18 AQ, S-5 μm, 4.6 mm ID×250 mm length (Shiseido, Japan)
Column temperature: Constant temperature around 35° C.
Detection wavelength: 280 nm
Flow rate: 1.0 mL/min
Mobile phase:
Acetonitrile/water/1 M ammonium acetate (200:800:1, v/v/v)
Isocratic analysis
Analysis time: 10 min

As shown in FIG. 13, it is clear that the respective crystals of Examples 1 to 3, 5 and 7 to 10 are more excellent in solubility than the crystal form B.

1. A salt of 5-[2-amino-4-(2-furyl)pyrimidin-5-yl]-1-methylpyridin-2(1H)-one.

2. A crystal of a salt according to claim 1.

3. The crystal according to claim 2, wherein the salt is an inorganic acid salt.

4. The crystal according to claim 2, wherein the salt is an organic acid salt.

5. The crystal according to claim 2, wherein the salt is hydrochloride, and the crystal has a diffraction peak at a diffraction angle (2θ±0.2°) of 8.5° and/or 16.7° in a powder X-ray diffraction.

6. The crystal according to claim 2, wherein the salt is hydrobromate, and the crystal has a diffraction peak at a diffraction angle (2θ±0.2°) of 8.4° and/or 11.3° in a powder X-ray diffraction.

7. The crystal according to claim 2, wherein the salt is sulfate, and the crystal has a diffraction peak at a diffraction angle (2θ±0.2°) of 10.0° and/or 11.5° in a powder X-ray diffraction.

8. The crystal according to claim 2, wherein the salt is sulfate, and the crystal has a diffraction peak at a diffraction angle (2θ±0.2°) of 6.4° and/or 10.2° in a powder X-ray diffraction.

9. The crystal according to claim 2, wherein the salt is phosphate, and the crystal has a diffraction peak at a diffraction angle (2θ±0.2°) of 11.2° and/or 17.4° in a powder X-ray diffraction.

10. The crystal according to claim 2, wherein the salt is phosphate, and the crystal has a diffraction peak at a diffraction angle (2θ±0.2°) of 5.0° and/or 6.6° in a powder X-ray diffraction.

11. The crystal according to claim 2, wherein the salt is methanesulfonate, and the crystal has a diffraction peak at a diffraction angle (2θ±0.2°) of 9.6°, 15.3° and/or 19.2° in a powder X-ray diffraction.

12. The crystal according to claim 2, wherein the salt is ethanesulfonate, and the crystal has a diffraction peak at a diffraction angle (2θ±0.2°) of 7.6° and/or 15.4° in a powder X-ray diffraction.

13. The crystal according to claim 2, wherein the salt is benzenesulfonate, and the crystal has a diffraction peak at a diffraction angle (2θ±0.2°) of 9.2° and/or 18.5° in a powder X-ray diffraction.

14. The crystal according to claim 2, wherein the salt is p-toluenesulfonate, and the crystal has a diffraction peak at a diffraction angle (2θ±0.2°) of 6.3° and/or 8.0° in a powder X-ray diffraction.
